(19) United States
(12) Patent Application Publication
(10) Pub. No.: US 2012/0100229 A1
(43) Pub. Date: Apr. 26, 2012

(54) Treatment and Prevention of White Matter Injury with KATP Channel Activators

(75) Inventor: Scott Rivkees, Orange, CT (US)

(73) Assignee: Yale University

(21) Appl. No.: 13/260,516

(22) PCT Filed: Mar. 25, 2010
(86) PCT No.: PCT/US10/28705

§ 371 Date: Jan. 12, 2012

Related U.S. Application Data
(60) Provisional application No. 61/163,966, filed on Mar. 27, 2009.

(51) Int. Cl. A61K 33/06; A61K 31/4406; A61K 31/4025; A61K 31/167; A61K 31/353; A61P 25/18; A61K 38/18; A61K 33/00; A61K 31/573; A61P 25/00; A61P 25/28; A61K 31/549; A61K 31/522

(52) U.S. Cl. 424/682; 514/223.2; 514/353; 514/422; 514/625; 514/456; 514/223.5; 514/263.34; 514/7.7; 424/600; 514/171

(57) ABSTRACT

The present invention includes a method of treating or preventing a CNS white matter injury in a patient in need thereof. The invention also includes a method of stimulating proliferation of a CNS cell in a patient in need thereof.

### BACKGROUND OF THE INVENTION

Oligodendrocytes, also known as oligodendroglia, are a variety of neuroglia (a subtype of macroglia) and act as myelinating cells of the central nervous system (CNS). Their main function is the insulation of axons (the long projection of nerve cells) in the CNS (comprising the brain and spinal cord) of higher vertebrates. A single oligodendrocyte can extend its processes to 50 axons, wrapping around approximately 1 mm of myelin sheath around each axon.

Oligodendrocytes arise during development from oligodendrocyte precursor cells or pre-oligodendrocytes (PreOLs). In the mammalian forebrain, the majority of pre-oligodendrocytes arise during late embryogenesis and early postnatal development from cells of the subventricular zones of the lateral ventricles. Subventricular zones migrate away from germinal zones to populate both developing white and gray matter, where they differentiate and mature into myelin-forming oligodendrocytes. However, it is not clear whether all pre-oligodendrocytes undergo this sequence of events. It has been suggested that some pre-oligodendrocytes undergo apoptosis and others fail to differentiate into mature oligodendrocytes but persist in the adult brain as oligodendrocyte progenitors (also known as oligodendrocyte stem cells).

As part of the nervous system, oligodendrocytes are closely related to nerve cells and, like all other glial cells, oligodendrocytes provide a supporting role for neurons. Additionally, the nervous system of mammals depends crucially on myelin sheaths, which reduce ion leakage and decrease the capacitance of the cell membrane. Myelin also increases impulse speed as saltatory propagation of action potentials occurs at the nodes of Ranvier in between Schwann cells (of the peripheral nervous system) and oligodendrocytes (of the central nervous system). Oligodendrocytes provide the same functionality as the insulation on a household electrical wire.

Serious clinical disorders affect CNS white matter during early development. These conditions include periventricular white matter injuries (PWMIs) and periventricular leukomalacoa (PVL), which affects more than 20% of very low birth weight premature infants and is in part related to loss of pre-oligodendrocytes. Other diseases that result in injury to the oligodendroglial cells include demyelinating diseases, such as multiple sclerosis and leukodystrophies. Cerebral palsy due to PWMI or PVL is caused by damage to developing oligodendrocytes in the brain areas around the cerebral ventricles. Spinal cord injury also causes damage to oligodendrocytes. In cerebral palsy, spinal cord injury, stroke and possibly multiple sclerosis, oligodendrocytes are thought to be damaged by the release of toxic neurotransmitters and chemicals that include cytokines or hypoxia (low oxygen levels). Oligodendrocyte dysfunction may also be implicated in the pathophysiology of schizophrenia and bipolar disorder. Oligodendrocytes are also susceptible to infection by the JC virus, which causes progressive multifocal leukoencephalopathy (PML), a condition which specifically affects white matter, typically in immunocompromised patients.

There is thus a long felt need in the art for effective pharmacological approaches to protect and stimulate development of pre-oligodendrocytes. Such approaches would be useful in the treatment of diseases that cause injury to the CNS white matter, such as periventricular white matter injuries. The present invention meets this need.

### SUMMARY OF THE INVENTION

The invention includes a method of treating or preventing a CNS white matter injury in a patient in need thereof. The method comprises administering to the patient a therapeutically effective amount of a pharmaceutical composition comprising a K_{ATP} channel activator, whereby the method promotes myelination of the CNS white matter.

In one embodiment, the activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl) propanamide, N-((3S,4R)-6-cyano-3-hydroxy-2,2-dimethylchroman-4-yl)-N-hydroxyacetamide, and acceptable salts thereof. In another embodiment, the activator is 7-chloro-3-methyl-4H-1,2,4-benzothiadiazine 1,1-dioxide or an acceptable salt thereof. In yet another embodiment, the CNS white matter injury is selected from the group consisting of periventricular leukomalacica, periventricular white matter injury, demyelinating disease, cerebral palsy, spinal cord injury, stroke injury, schizophrenia, degenerative CNS disorder, and bipolar disorder. In yet another embodiment, the demyelinating disease is multiple sclerosis or leukodystrophy. In yet another embodiment, the CNS white matter injury is periventricular leukomalacica or periventricular white matter injury. In yet another embodiment, the CNS white matter injury is stroke injury. In yet another embodiment, the method further comprises administering to the patient a therapeutically effective amount of at least one additional compound known to treat the CNS white matter injury. In yet another embodiment, the at least one additional compound is selected from the group consisting of caffeine, erythropoietin, magnesium sulfate, oxygen gas, dexamethasone, prednisone, and hydrocortisone. In yet another embodiment, the patient is human. In yet another embodiment, the patient is a premature infant.

The invention also includes a method of stimulating proliferation of a CNS cell in a patient in need thereof. The method comprises administering to the patient a therapeutically effective amount of a pharmaceutical composition comprising a K_{ATP} channel activator, wherein the CNS cell is selected from the group consisting of pre-oligodendrocytes, oligodendrocyte stem cells and glia cells.

In one embodiment, the activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl) propanamide, N-((3S,4R)-6-cyano-3-hydroxy-2,2-dimethylchroman-4-yl)-N-hydroxyacetamide, and acceptable salts thereof. In another embodiment, the activator is 7-chloro-3-methyl-4H-1,2,4-benzothiadiazine 1,1-dioxide or an acceptable salt thereof. In yet another embodiment, the method further comprises administering to the patient a therapeutically effective amount of at least one additional compound known to stimulate proliferation of the CNS cell. In yet another embodiment, the at least one additional compound is selected from the group consisting of caffeine, erythropoietin, magnesium sulfate, oxygen gas, dexamethasone, prednisone, and hydrocortisone. In yet another embodiment, the patient is human. In yet another embodiment, the patient is a premature infant.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the structures of various K_{ATP} channel activators.

FIG. 2 is a bar graph illustrating the degree of K_{ATP} channel activation by various chemical compounds in percent of the fluorescence intensity measured for the chemical compounds illustrated in FIG. 1 relative to control.

FIG. 3 illustrates the analysis of gene expression in oligodendrocytes by PCR. cDNA was prepared from the isolated mRNA of oligodendrocytes and used in PCR reactions containing primers specific to a specified gene (the genes analyzed are annotated on top of the figure). The ladder bands located on the first lane on the left were molecular weight markers. The white bands observed in the kir6.1 (SEQ ID NO:3), kir6.2 (SEQ ID NO:4), sur1 (SEQ ID NO:1), and sur2 (SEQ ID NO:2) lanes were amplified DNA obtained with the corresponding primers.

FIG. 4, comprising FIGS. 4A-4D, illustrates the analysis of protein expression in oligodendrocytes using immunochemistry and Western blotting.

FIGS. 4A and 4C illustrate A2B5-positive cells. FIGS. 4B and 4D illustrate O1-positive cells. FIGS. 4A and 4B were stained for Kir6.1. FIG. 4C was stained for A2B5, and FIG. 4D was stained for O1.

FIG. 5 illustrates Western blotting experiments performed with whole brain lysates (labeled as “BR”) and isolated pre-oligodendrocytes (labeled as “OL”). The antisera used are identified on the top of each lane.

FIG. 6, comprising FIGS. 6A-6B, illustrates effects of diazoxide on hypoxia-induced periventricular white matter injury. FIG. 6A illustrates the measured ventricle volume for treatment with vehicle or diazoxide. FIG. 6B illustrates brain ventricle observed on the course of the experiment.

FIG. 7, comprising FIGS. 7A-7B, illustrates the myelination of mouse brain under different treatment conditions, as evidenced by myelin-basic protein (MBP) labeling. FIG. 7A illustrates quantitative assessment of labeling using Image J Version 1.42q (National Institutes of Health, Bethesda Md.) at the mid-level of the corpus callosum. Data shown are mean±SEM from one experiment with 3-5 animals per treatment group and are representative of one other separate study performed at a different time. Three to six animals were used in each treatment group. *p<0.01, ANOVA. Level of background intensity is 10. FIG. 7B illustrates coronal images at level of corpus callosum for 4-6 animals per treatment group. MBP staining was performed at the same time. The box illustrates region of corpus callosum where labeling intensity was assessed. Photographs were taken at identical exposures.

### DETAILED DESCRIPTION OF THE INVENTION

The present invention includes a method of treating or preventing a CNS white matter injury in a patient in need thereof. The method comprises administering to the patient a therapeutically effective amount of a composition comprises a K_{ATP} channel activator, whereby the K_{ATP} channel activator stimulates the proliferation of pre-oligodendrocytes, leading to formation of myelinating oligodendrocytes.

The present invention also includes a method of stimulating proliferation of a CNS cell in a patient in need thereof. In one aspect, the cell is selected from the group consisting of pre-oligodendrocytes, oligodendrocyte stem cells and glia cells.

### DEFINITIONS

The definitions used in this application are for illustrative purposes and do not limit the scope used in the practice of the invention.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations off ±20% or ±10%, more preferably ±5%, even more preferably ±1%, and still more preferably ±0.1% from the specified value, as such variations are appropriate to perform the disclosed methods.

As used herein, the term “CNS” refers to central nervous system.

As used herein, an “ATP-sensitive potassium channel” or “K_{ATP} channel” is a type of potassium channel that is gated by ATP.

As used herein, the term “K_{ATP} channel activator” refers to a chemical compound that interacts with a K_{ATP} channel and (a) increases the baseline activity of the K_{ATP} channel or (b) increases the activity that the K_{ATP} channel has while another compound is bound to the channel.

As used herein, the term “diazoxide” refers to 7-chloro-3-methyl-4H-1,2,4-benzothiadiazine 1,1-dioxide; the term “P1075” refers to (E)-1-cyano-2-tert-pentyl-3-(pyridin-3-yl)guanidine; the term “levcromakalim” refers to (3S,4R)-3-hydroxy-2,2-dimethyl-4-(2-oxopyrrolidin-1-yl)chroman-6-carbonitrile; the term “pinacidil” refers to (E)-1-(3,3-dimethylbutan-2-yl)-2-cyano-3-(pyridin-4-yl)guanidine; the term “ZM226600” refers to 3,3,3-trifluoro-2-hydroxy-2-methyl-N-(4-(phenylsulfonyl)phenyl) propanamide; and the term “Y26763” refers to N-((3S,4R)-6-cyano-3-hydroxy-2,2-dimethylchroman-4-yl)-N-hydroxyacetamide.

As used herein, the language “acceptable salt” refers to a salt of the administered compounds prepared from pharmaceutically acceptable non-toxic acids including inorganic acids, organic acids, solvates, hydrates, or clathrates thereof.

As used herein, the term “polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides. The term “peptide” typically refers to short polypeptides.

As used herein with respect to the compounds useful within the methods of the invention, the term “biologically active” means that the compound elicits a biological response in a subject that may be monitored and characterized in comparison with an untreated subject. One possible biological response within the invention relates to the ability of the compound to treat a CNS white matter injury in a patient in need thereof. Another possible biological response within the invention relates to the ability of the compound to stimulate proliferation of a CNS cell in a patient in need thereof, wherein the cell is selected from the group consisting of pre-oligodendrocytes, oligodendrocyte stem cells and glia cells. Yet another possible biological response within the invention relates to the ability of the compound to treat hypoxic injury in a patient in a subject. These exemplified biological responses do not limit or restrict the disclosures or embodiments of the invention in any way.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, the term “medical intervention” means a set of one or more medical procedures or treatments that are required for ameliorating the effects of, delaying, halting or reversing a disease or disorder of a subject. A medical intervention may involve surgical procedures or not, depending on the disease or disorder in question. A medical intervention may be wholly or partially performed by a medical specialist, or may be wholly or partially performed by the subject himself or herself, if capable, under the supervision of a medical specialist or according to literature or protocols provided by the medical specialist.

As used herein, the term “subject” or “patient” refers to a human or a non-human mammal. Non-human mammals include, for example, livestock and pets, such as ovine, bovine, porcine, canine, feline and murine mammals. In one embodiment, the subject is canine, feline or human. In another embodiment, the subject is human. In yet another embodiment, the subject is a premature infant. In yet another embodiment, the subject is a premature infant born less than 35 weeks post-conception.

As used herein, the term “effective amount” or “therapeutically effective amount” refers to a non-toxic but sufficient amount of the composition used in the practice of the invention that is effective to, in non-limiting examples, treat a CNS white matter injury in a patient; or stimulate proliferation of a CNS cell in a patient, wherein the CNS cell is selected from the group consisting of pre-oligodendrocytes, oligodendrocyte stem cells and glia cells. The desired treatment may be prophylactic and/or therapeutic. That result may be reduction and/or alleviation of the signs, symptoms, or causes of a disease or disorder, or any other desired alteration of a biological system. An appropriate therapeutic amount in any individual case may be determined by one of ordinary skill in the art using routine experimentation.

The term “synergistic,” when applied to the use of at least two drugs in a therapeutic treatment, indicates that the therapeutic benefit obtained by combining the two or more drugs in a treatment is greater than the juxtaposition of the therapeutic benefit obtained when each drug is used by itself If the first drug provides benefit “x” and the second drug provides benefit “y”, the benefit provided by combining the two drugs has to be greater than “x+y” to characterize synergy or synergistic properties. Synergistic drugs may be administered concomitantly or sequentially, in the same formulation or different formulations.

A “therapeutic” treatment indicates a treatment administered to a subject who exhibits signs of pathology of a disease or disorder for the purpose of diminishing or eliminating those signs.

A “prophylactic” or “preventive” treatment indicates a treatment administered to a subject who does not exhibit signs of a disease or disorder or exhibits only early signs of the disease or disorder for the purpose of decreasing the risk of developing pathology associated with the disease or disorder.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids and bases, including inorganic acids, inorganic bases, organic acids, inorganic bases, solvates, hydrates, and clathrates thereof.

As used herein, the term “pharmaceutical composition” refers to a mixture of at least one compound of the invention with a pharmaceutically acceptable carrier. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, ameliorating, or preventing shivering in a subject.

As used herein, the term “applicator” is used to identify any device including, but not limited to, a hypodermic syringe, a pipette, and the like, for administering the compounds and compositions used in the practice of the invention.

### Methods of the Invention

In one aspect, the invention relates to the discovery that oligodendrocytes, oligodendrocyte stem cells and glia cells have K_{ATP} channels containing sulfonyl urea receptor (SUR) components. In another aspect, the invention relates to the discovery that treatment of oligodendrocytes, oligodendrocyte stem cells and glia cells with K_{ATP} channel activators stimulates the growth of the cells and promotes myelination. This effect may be used to overcome adverse effects of hypoxia-induced brain injury.

ATP-sensitive potassium channels are generally composed of eight protein subunits: four Kir6.x-type subunits and four sulfonylurea receptor (SUR) subunits, along with additional components. The Kir subunits have two transmembrane spans and form the channel's pore. The SUR subunits have three additional transmembrane domains, and contain two nucleotide-binding domains on the cytoplasmic side.

Four genes have been identified as members of the K_{ATP} gene family. The sur1 (SEQ ID NO:1) and kir6.2 (SEQ ID NO:4) genes are located in chr11p15.1, while kir6.1 (SEQ ID NO:3) and sur2 (SEQ ID NO:2) genes reside in chr12p12.1. The kir6.1 and kir6.2 genes encode the pore-forming subunits of the K_{ATP} channel, with the SUR subunits being encoded by the sur1 (SUR1) gene or selective splicing of the sur2 gene (SUR2A and SUR2B).

In one embodiment, administering K_{ATP} channel activators to oligodendrocytes is useful for the treatment or prevention of periventricular white matter injuries. Considering that there are more than 100,000 premature infants born each year in the United States at risk for of periventricular white matter injuries, the methods disclosed in the present invention have broad commercial potential and clinical utility.

In one embodiment, the invention includes a method of treating or preventing a CNS white matter injury in a patient in need thereof. The method comprises administering to the patient a therapeutically effective amount of a pharmaceutical composition comprising a K_{ATP} channel activator. The method promotes myelination of the CNS white matter. In another embodiment, the activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl)propanamide, N-((3S,4R)-6-cyano-3-hydroxy-2,2-dimethylchroman-4-yl)-N-hydroxyacetamide, and acceptable salts thereof. In yet another embodiment, the activator is 7-chloro-3-methyl-4H-1,2,4-benzothiadiazine 1,1-dioxide or an acceptable salt thereof. In yet another embodiment, the CNS white matter injury is selected from the group consisting of periventricular leukomalacica, periventricular white matter injury, demyelinating disease, cerebral palsy, spinal cord injury, stroke injury, schizophrenia, degenerative CNS disorder, and bipolar disorder. In yet another embodiment, the demyelinating disease is multiple sclerosis or leukodystrophy. In yet another embodiment, the CNS white matter injury is periventricular leukomalacica or periventricular white matter injury. In yet another embodiment, the CNS white matter injury is stroke injury. In yet another embodiment, the degenerative CNS disorder is Alzheimer's disease or multiple sclerosis. In yet another embodiment, the patient is human. In yet another embodiment, the patient is a premature infant.

In one embodiment, the invention includes a method of stimulating proliferation of a CNS cell in a patient in need thereof. The method comprises administering to the patient a therapeutically effective amount of a pharmaceutical composition comprising a K_{ATP} channel activator. The CNS cell is selected from the group consisting of pre-oligodendrocytes, oligodendrocyte stem cells and glia cells. In another embodiment, the activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl)propanamide, N-((3S,4R)-6-cyano-3-hydroxy-2,2-dimethylchroman-4-yl)-N-hydroxyacetamide, and acceptable salts thereof. In yet another embodiment, the activator is 7-chloro-3-methyl-4H-1,2,4-benzothiadiazine 1,1-dioxide or an acceptable salt thereof. In yet another embodiment, the patient is human. In yet another embodiment, the patient is a premature infant.

### Compounds Useful in the Methods of the Invention

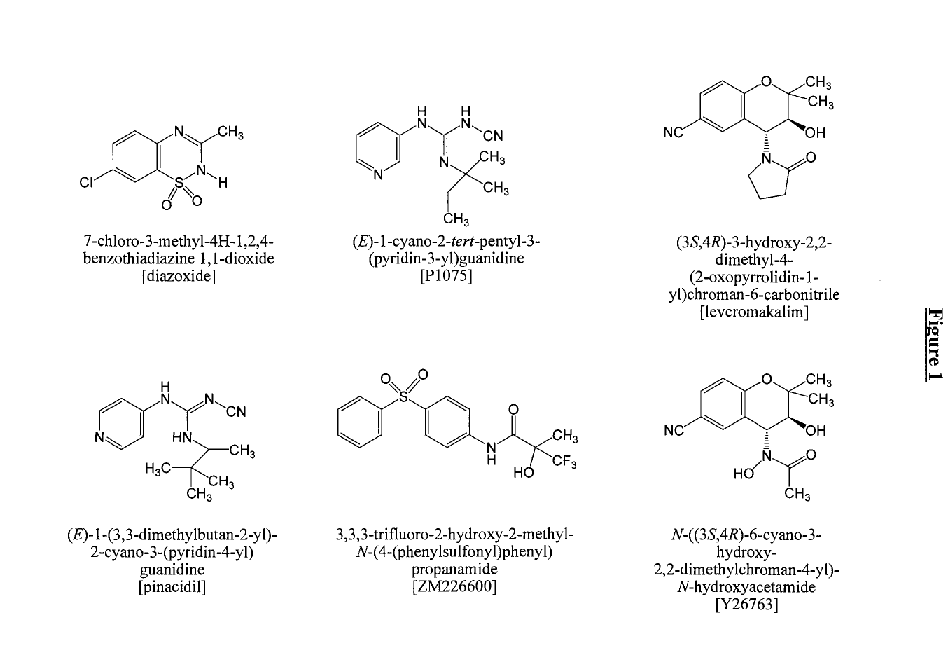

Non-limiting examples of the compounds useful within the invention are illustrated in FIG. 1. Non-limiting examples of compounds are 7-chloro-3-methyl-4H-1,2,4-benzothiadiazine 1,1-dioxide (also known as diazoxide), (E)-1-cyano-2-tert-pentyl-3-(pyridin-3-yl)guanidine (also known as P1075), (3S,4R)-3-hydroxy-2,2-dimethyl-4-(2-oxopyrrolidin-1-yl)chroman-6-carbonitrile (also known as levcromakalin), (E)-1-(3,3-dimethylbutan-2-yl)-2-cyano-3-(pyridin-4-yl)guanidine (also known as pinacidil), 3,3,3-trifluoro-2-hydroxy-2-methyl-N-(4-(phenylsulfonyl)phenyl)propanamide (also known as ZM226600), and N-((3S,4R)-6-cyano-3-hydroxy-2,2-dimethylchroman-4-yl)-N-hydroxyacetamide (also known as Y26763).

The compounds useful within the methods of the present invention may be obtained from commercial sources (Sigma-Aldrich, St. Louis, Mo.) or prepared according to methods known to those skilled in the art.

7-Chloro-3-methyl-4H-1,2,4-benzothiadiazine 1,1-dioxide may be prepared by reacting 2,4-dichloronitrobenzene and benzylthiol to yield 4-chloro-2-benzylthionitrobenzene. Chlorination of this compound, followed by treatment with ammonia, yields 5-chloro-2-nitrobenzenesulfonamide. Reduction with tin and HCl yields 2-amino-5-chloro-benzenesulfonamide, which is condensed with ethylacetate to yield 7-chloro-3-methyl-4H-1,2,4-benzothiadiazine 1,1-dioxide.

Salts of Compounds Useful within the Methods of the Invention

The compounds useful within the methods of the invention may form salts with acids or bases, and such salts are included in the present invention. The preferred salts are pharmaceutically-acceptable salts. The term “salts” embraces addition salts of free acids or free bases that are compounds useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the methods of the present invention, such as for example utility in process of synthesis, purification or formulation of compounds.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base with the compound.

### Pharmaceutical Compositions

When used in vivo, a compound useful within the invention is preferably administered as a pharmaceutical composition, comprising a compound useful within the invention and a pharmaceutically acceptable carrier. The compounds may be present in a pharmaceutical composition in an amount from 0.001 to 99.9 wt %, more preferably from about 0.01 to 99 wt %, and even more preferably from 0.1 to 95 wt %.

Single or multiple administrations of the compositions may be administered depending on the dosage and frequency as required and tolerated by the subject. In any event, the administration regime should provide a sufficient quantity of the composition of this invention to treat the subject effectively.

All of the various compounds useful within the methods of the present invention to be administered need not be administered together in a single composition. The different compounds may be administered in separate compositions. For example, if three different compounds useful within the methods of the invention are to be administered, the three compounds may be delivered in three separate compositions. In addition, each compound may be delivered at the same time, or the compounds may be delivered consecutively with respect to one another. Thus, the mixture of the compounds useful within the methods of the invention may be administered in a single composition, or in multiple compositions comprising one or more compounds useful within the methods of the invention.

A mixture of two or more compounds useful within the methods of the invention may be administered in equimolar concentrations to a subject in need of such treatment. In another instance, two or more of the compounds are administered in concentrations that are not equimolar. In other instances, two or more of the compounds are administered as equal amounts, by weight, per kilogram of body weight. For example, the compounds may be administered in equal amounts, based on the weight of the subject. In another instance, the compounds are administered in unequal amounts. In yet other instances, the amount of each compound to be administered is based on its biological activity. In general, the schedule or timing of administration of a pharmaceutical composition of the invention is according to the accepted practice for the procedure being performed.

The regimen of administration may affect what constitutes an effective amount. For example, the therapeutic formulations may be administered to the subject prior to, during or after the onset of a CNS white matter injury. Further, several divided dosages, as well as staggered dosages, may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In view of the disclosure contained herein, those skilled in the art will appreciate that the present compositions are capable of having a beneficial effect in a variety of surgical interventions involving the CNS. It is therefore contemplated that the compositions of this invention may take numerous and varied forms, depending upon the particular circumstance of each application. For example, the compounds may be incorporated into a solid pill or in the form of a liquid dispersion or suspension. In general, therefore, the compositions of the present invention preferably comprise a compound useful within the invention and a suitable, non-toxic, physiologically acceptable carrier. The compounds may be administered by any method designed to allow compounds to have a physiological effect. Administration may occur enterally or parenterally; for example orally, rectally, intracisternally, intravaginally, intraperitoneally or locally. Parenteral and local administrations are preferred.

For some applications involving treatment or prevention of CNS white matter injury in the broadest sense, it is desirable to have available a physically applicable or implantable predetermined solid form of material containing the composition of the invention. In such embodiments, the compositions of the present invention are preferably combined with a solid carrier that itself is bio-acceptable and suitably shaped for its use.

Formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods used in the practice of the invention may be prepared, packaged, or sold in formulations suitable for oral, intracisternal, intraperitoneal or local, or another route of administration. Other contemplated formulations include projected nanoparticles, liposomal preparations, resealed erythrocytes containing the active ingredient, and immunologically-based formulations.

The pharmaceutical compositions of the invention may be dispensed to the subject under treatment with the help of an applicator. The applicator to be used may depend on the specific medical condition being treated, amount and physical status of the pharmaceutical composition, and choice of those skilled in the art.

The pharmaceutical compositions of the invention may be provided to the subject or the medical professional in charge of dispensing the composition to the subject, along with instructional material. The instructional material includes a publication, a recording, a diagram, or any other medium of expression, which may be used to communicate the usefulness of the composition and/or compound used in the practice of the invention in a kit. The instructional material of the kit may, for example, be affixed to a container that contains the compound and/or composition used in the practice of the invention or shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### Oral Administration.

A formulation of a pharmaceutical composition used in the practice of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. As used herein, an “oily” liquid comprises a carbon-containing liquid molecule that exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture.

Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition used in the practice of the invention that are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropyl methylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Powdered and granular formulations of a pharmaceutical preparation used in the practice of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition used in the practice of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination thereof. The compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (e.g. such as with a physiologically degradable material), and methods of absorbing an aqueous or oily solution or suspension into an absorbent material, with or without subsequent drying.

### Parenteral Administration.

As used herein, “parenteral administration” of a pharmaceutical composition includes any route of administration characterized by physical breaching of a tissue of a subject and administration of the pharmaceutical composition through the breach in the tissue. For parenteral administration, the compounds may be formulated for injection or infusion, for example, intravenous, intracranial, intraspinal, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents, may be used. Such formulations may further comprise one or more additional ingredients including, but not limited to, suspending, stabilizing, or dispersing agents. In one embodiment of a formulation for parenteral administration, the active ingredient is provided in dry (e.g. powder or granular) form for reconstitution with a suitable vehicle (e.g. sterile pyrogen free water) prior to parenteral administration of the reconstituted composition.

Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraspinal, intracranial, subcutaneous, intraperitoneal, intramuscular, intrasternal injection, and kidney dialytic infusion techniques. Particularly preferred parenteral administration methods include intravascular administration (e.g., intravenous bolus injection, intravenous infusion, intra-arterial bolus injection, intra-arterial infusion and catheter instillation into the vasculature), peri- and intratarget tissue injection, subcutaneous injection or deposition including subcutaneous infusion (such as by osmotic pumps), intramuscular injection, intraperitoneal injection, and direct application to the target area, for example by a catheter or other placement device.

For parenteral administration, the compositions for administration may commonly comprise a solution or suspension of the compound in a pharmaceutically acceptable carrier, preferably an aqueous carrier. A variety of aqueous carriers may be used, e.g., buffered saline and the like. These suspensions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The amount of the compound may vary widely, and will be selected primarily based on fluid volumes, viscosities, body weight and the like in accordance with the particular mode of administration selected and the subject's needs. A typical pharmaceutical composition for intravenous administration would be about 1 to 3,000 mg per subject per day. Dosages from 1 up to about 1,000 mg per subject per day may be used, particularly when the drug is administered to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., 1980, Mack Publishing Company, Easton (PA).

### Transmucosal Administration.

Transmucosal administration is carried out using any type of formulation or dosage unit suitable for application to mucosal tissue. For example, the selected active agent may be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethral Administration.

With regard to transurethral administration, the formulation may comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer may be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecyl-cyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

### Intranasal or Inhalation Administration.

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators may be used. For liquid formulations, the active agent may be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers may also include nasal gels, creams, pastes or ointments with a viscosity of e.g., from about 10 to about 6,500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, may also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation may also comprise dry powder formulations, particularly insufflations in which the powder has an average particle size of from about 0.1 μm to about 50 gm, e.g., from about 1 μm to about 25 μm.

### Topical Formulations.

Topical formulations may be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

### Transdermal Administration.

Transdermal compound administration, which is known to one skilled in the art, involves the delivery of pharmaceutical compounds via percutaneous passage of the compound into the systemic circulation of the subject. Topical administration may also involve the use of transdermal administration such as transdermal patches or iontophoresis devices. Other components may be incorporated into the transdermal patches as well. For example, compositions and/or transdermal patches may be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions may include creams, sprays, lotions, gels, ointments, eye drops, nose drops, ear drops, and the like. In such dosage forms, the compositions of the invention may be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions may contain polyethylene glycol 400. They may be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, may be impregnated with the compositions in solution, lotion, cream, ointment or other such form may also be used for topical application. The compositions may also be applied topically using a transdermal system, such as one of an acrylic-based polymer adhesive with a resinous crosslinking agent impregnated with the composition and laminated to an impermeable backing.

Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir that, in this case, may be either a polymeric matrix as described above, or be a liquid or hydrogel reservoir, or take some other form.

### Intrathecal Administration.

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication may then be administered directly to cells in the spinal cord involved in conveying sensory and motor signals associated with lower urinary tract disorders.

### Intravesical Administration.

The term intravesical administration is used herein in its conventional sense to mean delivery of a drug directly into the bladder. Suitable methods for intravesical administration may be found, for example, in U.S. Pat. Nos. 6,207,180 and 6,039,967.

### Additional Administration Forms.

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Application Nos. 2003/0147952, 2003/0104062, 2003/0104053, 2003/0044466, 2003/0039688, and 2002/0051820. Additional dosage forms of this invention also include dosage forms as described in PCT Application Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems.

In certain embodiments, the formulations of the present invention may be, but are not limited to, short-term release or rapid-offset release, as well as controlled release, for example, sustained release, delayed release and pulsatile release formulations.

The term short-term or rapid-offset release is used in its conventional sense to refer to a drug formulation that provides for release of the drug immediately after drug administration.

As used herein, short-term or rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments there between after drug administration after drug administration.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be longer than the time required for the release of the same amount of agent administered in bolus form.

For sustained release, the compounds may be formulated with a suitable polymer or hydrophobic material that provides sustained release properties to the compounds. As such, the compounds may be administered in the form of microparticles for example, by injection or in the form of wafers or discs by implantation.

In a preferred embodiment of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

In a preferred embodiment of the invention the compounds useful within the invention are administered to a subject, alone or in combination with another pharmaceutical agent, using a delayed release formulation.

The term pulsatile release is used herein in its conventional sense to refer to a drug formulation that provides release of the drug in such a way as to produce pulsed plasma profiles of the drug after drug administration.

In a preferred embodiment of the invention, the compounds useful within the invention are administered to a subject, alone or in combination with another pharmaceutical agent, using a pulsatile release formulation.

### Dosing

One skilled in the art may readily determine an effective amount of compounds useful within the methods of the invention to a given subject, by taking into account factors such as the size and weight of the subject; the extent of CNS white matter injury or the extent of intended cell proliferation or the extent of damage in CNS cells caused by hypoxia in the subject, the age, health and sex of the subject; the route of administration; and whether the administration is local or systemic. Generally, the amount of compounds to be administered to a subject depends upon the degree of CNS white matter injury, and the biological activity exhibited by the compounds useful within the invention. Those skilled in the art may derive appropriate dosages and schedules of administration to suit the specific circumstances and needs of the subject. For example, suitable doses of compounds useful within the invention to be administered may be between about 0.015 mg/kg and about 45 mg/kg body weight. In some embodiments, dosages are between about 0.1 mg/kg and about 20 mg/kg body weight.

It is understood that the effective dosage will depend on the age, sex, health, and weight of the subject, kind of concurrent treatment, if any, frequency of treatment, and the nature of the effect desired. The most preferred dosage will be tailored to the individual subject, as is understood and determinable by one of skill in the art, without undue experimentation.

A medical doctor, e.g., physician or veterinarian, having ordinary skill in the art may readily determine and prescribe the effective amount of the pharmaceutical composition required. For example, the physician or veterinarian could start doses of the compounds useful within the invention employed in the pharmaceutical composition at levels lower than that required in order to achieve the desired therapeutic effect and gradually increase the dosage until the desired effect is achieved.

A suitable dose of a compound of the present invention may be in the range of from about 1 mg to about 3,000 mg per day, such as from about 10 mg to about 2,000 mg, for example. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12 hour interval between doses.

In some embodiments, dose of a compound useful within the invention is between about 1 mg and about 2,000 mg. In some embodiments, a dose of a compound useful within the invention used in compositions described herein is between about 2 mg and about 1,000 mg. In some embodiments, a dose of a compound useful within the invention used in compositions described herein is between about 4 mg and about 500 mg. In some embodiments, a dose of a compound useful within the invention used in compositions described herein is between about 8 mg and about 250 mg. In some embodiments, a dose of a compound useful within the invention used in compositions described herein is between about 16 mg and about 125 mg. In some embodiments, a dose of a compound useful within the invention used in compositions described herein is between about 30 mg and about 60 mg, and any and all whole or partial increments there between.

The amount of compound dosed per day may be administered every day, every other day, every 2 days, every 3 days, every 4 days, every 5 days, etc.

The pharmaceutical compositions for use in the method of the invention may be prepared, packaged, formulated or sold in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose. The specifications for the dosage unit forms of the invention are dictated by and directly dependent on: (a) the unique characteristics of the therapeutic compound and the particular therapeutic effect to be achieved, and (b) the limitations inherent in the art of compounding/formulating such a therapeutic compound for the treatment of shivering or temperature spiking in a subject.

### Combination Therapies and Treatments

The compounds of the present invention may be useful for the methods of the present invention in combination with a therapeutically effective amount of at least one additional compound known to treat CNS white matter injuries or stimulate the growth of CNS cells. The additional compound or compounds may comprise compounds of the present invention or other compounds known to prevent or treat CNS white matter injuries or stimulate the growth of CNS cells. Non-limiting examples of compounds known to prevent or treat CNS white matter injuries or stimulate the growth of CNS cells are caffeine, erythropoietin, magnesium sulfate, oxygen gas, dexamethasone, prednisone and hydrocortisone.

In an embodiment of this aspect of the present invention, the therapeutic effect achieved by the combination above is synergistic, in that, the therapeutic effect of the combination is greater than the sum of the therapeutic effect achieved by the separate administration of the compounds disclosed within the invention and the at least one additional compound known to treat CNS white matter injuries or stimulate the growth of CNS cells.

A combination of compounds described herein may either result in synergistic increase in effectiveness against CNS white matter injuries, relative to effectiveness following administration of each compound when used alone, or such an increase may be additive. Compositions described herein typically include lower dosages of each compound in a composition, thereby avoiding adverse interactions between compounds and/or harmful side effects, such as ones that have been reported for similar compounds. Furthermore, normal amounts of each compound when given in combination could provide for greater efficacy in subjects who are either unresponsive or minimally responsive to each compound when used alone.

A synergistic effect may be calculated, for example, using suitable methods such as, for example, the Sigmoid-Emax equation (Holford and Scheiner, 1981, Clin. Pharmacokinet. 6: 429-453), the equation of Loewe additivity (Loewe and Muischnek, 1926, Arch. Exp. Pathol Pharmacol. 114: 313-326) and the median-effect equation (Chou and Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Chemicals.

Chemicals were purchased from Sigma-Aldrich (Saint Louis, Mo.).

### Determination of Cell Viability and Toxicity.

Cell viability and toxicity were determined using the LIVE/DEAD® viability/cytotoxicity assay kit for mammalian cells (InVitrogen, Carlsbad, Calif.). Live cells were identified by the conversion of the non-fluorescent cell-permeant calcein AM to the fluorescent calcein (excitation: 495 nm; emission: 515 nm) by intracellular esterase activity of viable cells. At the same time, dead cells were identified by uptake of the ethidium homodimer-1 (EthD-1; excitation: 495 nm; emission: 635 nm), which is excluded by the intact plasma membrane of live cells, but enters cells with damaged membranes and binds to nucleic acids. This assay has been used successfully for assessment of cell death in numerous studies including oligodendrocyte cell death/viability (Sontheimer et al., 1994, J. Neurosci. 14:2464-75; Cubells et al., 1994, J. Neurosci. 14:2260-71; Larocca et al., 1997, Neurochem. Res. 22:529-34; Pang et al., 2000, J. Neurosci. Res. 62:510-20).

### Example 1

### Concentration-Response Studies of K_{ATP} Channel Activators

Purified oligodendrocyte progenitor cell cultures were prepared as described (Othman et al., 2003, Glia 44:166-172). In brief, primary rat mixed glial cell cultures were isolated from whole brains of postnatal day (P) 1 rats, dissociated into single cells, and cultured into poly-D-lysine (PDL, Sigma-Aldrich, St. Louis, Mo.) coated T75 tissue culture flasks. Plating medium consisted of Dulbecco's modified Eagle's medium (DMEM, Invitrogen, Carlsbad, Calif.) supplemented with 10% fetal bovine serum (FBS; InVitrogen, Carlsbad, Calif.), 2 mM L-glutamine, 100 μM streptomycin, and 10 μg/ml penicillin. Tissue cultures were maintained at 37° C. in a humidified 5% CO₂ incubator, and medium was exchanged every 3 days. Once confluent (after 7-9 days), microglia were separated by mechanical shaking of flasks on a rotary shaker for 60 min at 200 rpm and removed. After addition of fresh medium, the remaining cells were allowed to recover overnight before repeating the mechanical shaking for an additional 16 h at 200 rpm to isolate oligodendrocyte progenitor cells. To ensure purity of oligodendrocyte progenitor cell cultures, the isolated cells were transferred to a tissue culture dish, from which the loosely attached oligodendrocyte progenitor cells were detached by gentle shaking after 60 min, leaving behind attached microglia and astrocytes. Oligodendrocyte progenitor cells were plated onto PDL coated 96 well plates using an automated dispenser and allowed to adhere to the plates over the next 1-2 days. This procedure yielded 98% A2B5-positive (OPC marker), and 2% MBP-positive (mature OL marker) cells. GFAP-positive (astrocyte marker) or O22A-positive (microglia marker) cells could not be detected in cultures prepared in this manner.

Cells were exposed to various K_{ATP} channel stimulators, with concentrations of compounds ranging from 1 nM to 100 μM, for 72-96 h. Pre-oligodendrocytes were cultured in 384-well plates, at a concentration of 10,000 cells per well. Cultures were washed two times with DMEM to remove any residual serum and then the respective solutions of K_{ATP} channel activators were added daily in an incubation buffer of DMEM with 0.5% FBS+10 μg/ml biotin and N₂ supplement. Cells were then placed at 37° C. in a humidified atmosphere containing 5% CO₂. All concentrations were tested in triplicate and each study was repeated twice.

To evaluate cell proliferation in response to K_{ATP} channel stimulators, the CyQUANT® NF Cell Proliferation assay (Invitrogen, Carlsbad, Calif.) was used according to the manufacturer's instructions. This assay measured cellular DNA content as a direct index of cell proliferation. Since cellular DNA content is highly proportional to cell number, this is a very accurate way to assess cell proliferation specifically. At the end of drug exposure, medium was removed and a stock solution of the green-fluorescent CyQUANT GR dye (prepared according to manufacturer's instructions) was added. Upon binding to DNA, the GR dye shows a measurable enhancement in fluorescent intensity. Cells were returned to the incubator (37° C.) for 2 h, which resulted in maximal and stable changes in fluorescence. Fluorescence was measured using an Envision Multilabel reader (Perkin-Elmer; Excitation: 480 nm, Emission: 530 nm).

IC₅₀ values for each compound were then calculated. IC₅₀ value for diazoxide was 10 μM; for ZM26600, 2.5 μM; pinacidil, 10 μM; for Y26763, 200 nM; for levcromakalim, 2.5 μM; and for P 1075, 100 nM.

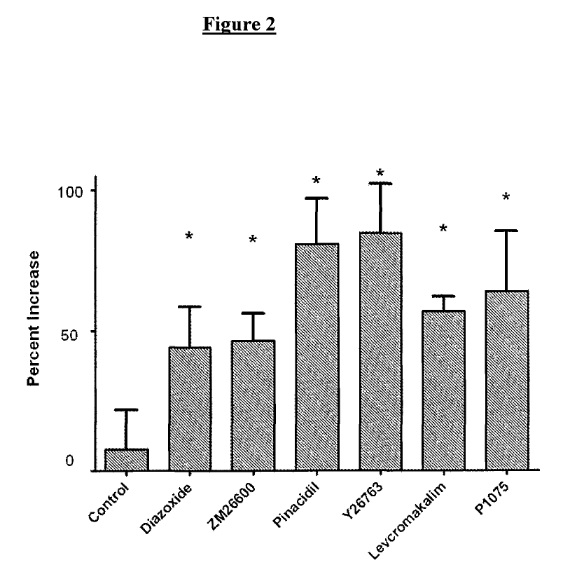

The results indicated that diazoxide, a known K_{ATP} channel activator, stimulated pre-oligodendrocyte proliferation (IC₅₀<100 nM) (FIG. 2).

Other commercially available compounds known to activate K_{ATP} channels were also tested at fixed doses, indicating potent stimulation of pre-oligodendrocyte proliferation. Therefore, the experiments showed that diazoxide and other K_{ATP} channel activators also stimulated pre-oligodendrocyte proliferation.

### Example 2

### Cell Toxicity Studies

Cell toxicity studies were performed with diazoxide in 384 well plates of cultured pre-oligodendrocytes. Pre-oligodendrocytes were plated at a density of 10,000 cells/well. The compound was added at concentrations 10, 50, 100 and 1000-fold greater than the observed EC₅₀ value (10 nM). All concentrations were tested in triplicate, and each study will be repeated twice.

Cell viability and toxicity was determined 48 hours later using the LIVE/DEAD® viability/cytotoxicity assay kit for mammalian cells (InVitrogen, Carlsbad, Calif.). The percentage of live and dead cells was then calculated and data were analyzed to determine the cytotoxicity index, LC₅₀.

Diazoxide was found not to be toxic to PreOLs even at 10 μM, a concentration that was 1000-fold greater than the concentration that stimulated pre-oligodendrocyte proliferation.

### Example 3

### Expression of SUR2 and KIR6.1 in OLs

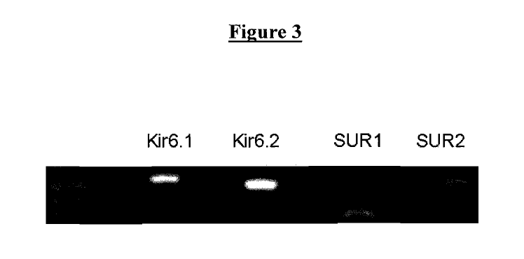

The expression of K_{ATP} channel components SUR and KIR6s was evaluated in oligodendrocytes by PCR. cDNA was made from oligodendrocyte precursor cells and mature oligodendrocytes. DNA was amplified from cells and used in PCR reactions with SUR or KIR6 subtype-specific primers. KIR6.1, KIR6.2, SUR1, and SUR2 mRNA expression was observed in pre-oligodendrocytes and mature oligodendrocytes (FIG. 3).

The expression of K_{ATP} channel components SUR and KIR6s was further evaluated using immunocytochemistry and Western blotting. For immunostaining studies, pre-oligodendrocytes were plated onto poly-D-lysine-coated 12-mm glass coverslips. Coverslips were probed with antisera against SUR1 and 2 and KIR6.1 and 6.2 (Santa Cruz Biotechnology, Santa Cruz, Calif.). The use of these antisera has been validated (Simard et al., 2007, J. Clin. Invest. 117: 2105-2113; Porksen et al., 2007, J. Clin. Endocrinol. Metab.; Simard et al., 2006, Nat. Med. 12(4):433-440; Jiang et al., 2006, Am. J. Physiol. Heart Circ. Physiol. 290(5):H1770-1776; Morrissey et al., 2005, BMC Physiol. 5(1): online; Jiang et al., 2006, Am. J. Physiol. Heart Circ. Physiol. 290:H1770-H1776; Morrissey et al., 2005, BMC Physiol. 5(1): online; Jin et al., 2004, Am. J. Physiol. Gastrointest. Liver Physiol. 287:G274-G285; Wang et al., 2003, Am. J. Physiol. Endocrinol. Metab. 284(5):E988-E1000).

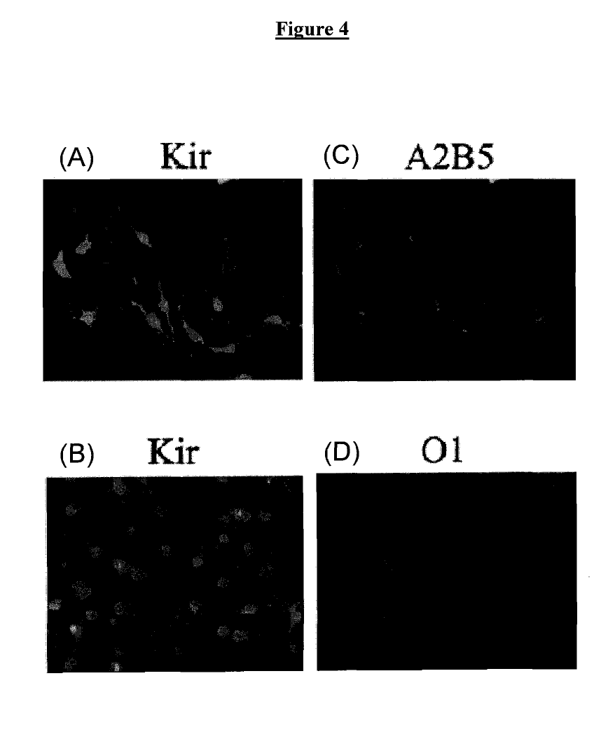

Florescent-labeled secondary antibodies were used to visualize the reaction product. Using microscopy, labeling of KIR6.1, KIR6.2 and SUR2 was observed in oligodendrocytes that were A2B5 and 01-positive (FIG. 4).

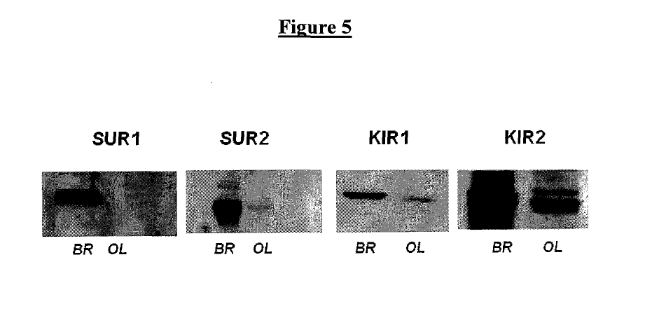

Expression of K_{ATP} channels in oligodendrocytes was further evaluated using Western blotting in whole brain lysates and pre-oligodendrocytes. Cells were plated on poly-D-lysine-coated 100 mm dishes. Cells were then lysed and proteins separated by gel electrophoresis. Blots were probed using SUR1 and 2 and KIR6.1 and 6.2 antisera. The reaction product was visualized using the ECL Chemoluminescent system (perkin-Elmer Life Sciences, Downers Grove, Ill.). When blots were examined, bands of the appropriate sizes for KIR6.1 (51 kDa) and 6.2 (40 kDa) and for Sur2 (180 kDa) were observed (FIG. 5).

Taken together, these experiments suggest that pre-oligodendrocytes express the SUR2, KIR6.1 and KIR6.2 genes and proteins, which are components of K_{ATP} channels.

### Example 4

### Protective Effects of K_{ATP} Channel Activators on Hypoxia-Induced Periventricular White Matter Injury

The effect of diazoxide in hypoxia-induced periventricular white matter injury was assessed. C57BL/6 mice were reared in room air of 10% O₂ from P3 to P12. Pups were treated with daily injection of either diazoxide or vehicle, with at least eight animals in each treatment group. Mice were weighed every two days. At P12, brains were removed. Ventricle size and myelin basic protein (MBP) labeling were assessed.

C57BL/6 mice were exposed to low or normal oxygen conditions from P3-P12 (Turner et al., 2003, Proc. Natl. Acad. Sci. USA 100:11718-11722). In brief, litters of pups (P3) were placed with the dam in a Plexiglas chamber, in which oxygen levels were maintained at 9.5±1.0%, O₂ levels were continuously monitored using a Cameron Instrument (Ontario, Canada) dual channel oxygen monitor attached to O₂ electrodes placed at each end of the chamber. Control animals were kept in room air (˜22%) outside the Plexiglas chamber. Animals were removed from the chamber daily for less than 15 min to allow for diazoxide (10 mg/kg i.p.) or vehicle administration, as well as observation of weight gain. Mice were euthanized at P12, brains were harvested and shock frozen in ice cold (−20° C.) 2-methylbutane and stored at −80° C. until assessment of ventricular size or MBP immunocytochemistry (see below). At least eight animals were studied in each treatment group.

Ventricle size was determined as reported (Turner et al., 2003, Proc. Natl. Acad. Sci. USA 100:11718-11722). Animals were weighed, anesthetized, and decapitated. Brains were shock frozen in ice cold (−20° C.) 2-methylbutane and stored at −80° C. Coronal sections spanning the brain were cut in a cryostat at a thickness of 16 μm in a Zeiss cryostat. Sections were mounted onto glass slides and stained with Phoenix Blue (Thermo Scientific, Waltham, Mass.). Serial sections through the midstriatum were photographed to include the lateral ventricle region. Ventricular sizes were quantified using Sigma Scan Pro Image Analysis Version 5.0.0 (SPSS Inc., Chicago, Ill.). The ventricular area, outlined by the Phoenix Blue staining, was measured and numerically integrated across the thickness of the slice. Images were obtained using a Leica florescence microscope.

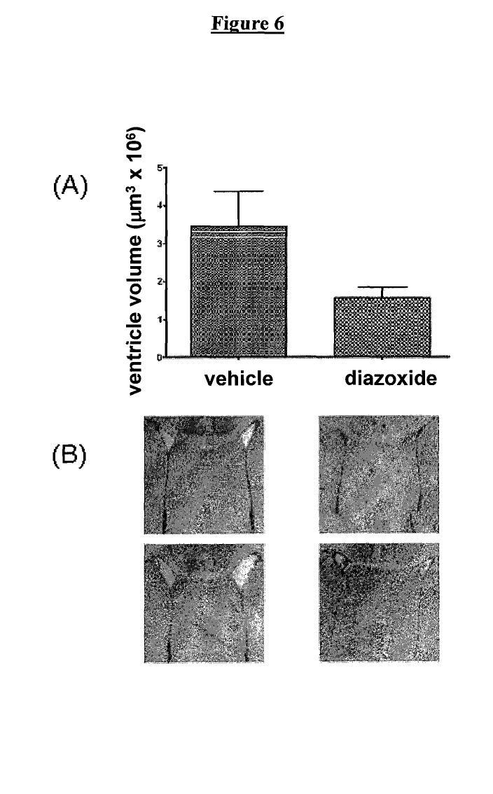

Upon sectioning of the mice brains, ventriculomegaly was observed in the hypoxia-vehicle group. In the hypoxia-diazoxide group, the ventricles were not enlarged (p<0.05; ANOVA) (FIG. 6). Mean±SD are shown.

The neonatal mice treated with diazoxide and reared in 10% O₂ did not show signs of ventriculomegaly or reduced myelination. This suggested that diazoxide may prevent periventricular white matter injury.

The experiments described above suggested that diazoxide and ATP channel activators estimulated pre-oligodendrocyte proliferation in vitro and myelination in vivo, consistent with the fact that pre-oligodendrocytes express K_{ATP} channels. Diazoxide was also shown to prevent hypoxia-induced ventriculomegaly and hypomyelination, which are characteristic features of periventricular white matter injury.

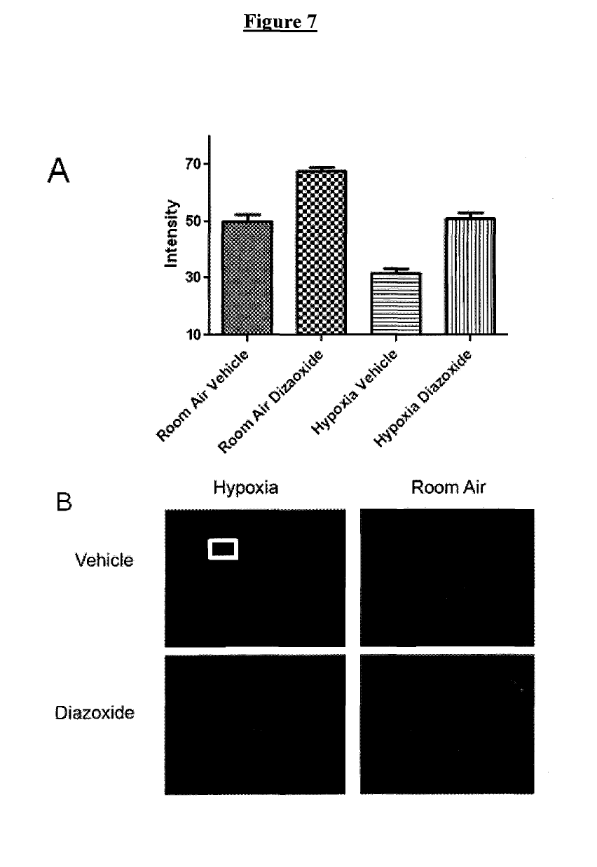

Animals reared in chronic hypoxia and room air demonstared increased myelination with diazoxide treatment, as illustrated in FIG. 7. Hypoxia caused diffuse reduction in cerebral myelin basic protein-labeling, which was markedly improved with diazoxide. More myelin basic protein-labeling was also observed in diazoxide-treatment mice reared in room air as compared to those treated with vehicle.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating or preventing a CNS white matter injury in a patient in need thereof, wherein said method comprises administering to said patient a therapeutically effective amount of a pharmaceutical composition comprising a K_{ATP} channel activator, whereby said method promotes myelination of said CNS white matter.

2. The method of claim 1, wherein said activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl)propanamide, N-((3S,4R)-6-cyano-3-hydroxy-2,2-dimethylchroman-4-yl)-N-hydroxyacetamide, and acceptable salts thereof.

3. The method of claim 2, wherein said activator is 7-chloro-3-methyl-4H-1,2,4-benzothiadiazine 1,1-dioxide or an acceptable salt thereof.

4. The method of claim 1, wherein said CNS white matter injury is selected from the group consisting of periventricular leukomalacica, periventricular white matter injury, demyelinating disease, cerebral palsy, spinal cord injury, stroke injury, schizophrenia, degenerative CNS disorder, and bipolar disorder.

5. The method of claim 4, wherein said demyelinating disease is multiple sclerosis or leukodystrophy.

6. The method of claim 4, wherein said CNS white matter injury is periventricular leukomalacica or periventricular white matter injury.

7. The method of claim 4, wherein said CNS white matter injury is stroke injury.

8. The method of claim 1, further comprising administering to said patient a therapeutically effective amount of at least one additional compound known to treat said CNS white matter injury.

9. The method of claim 8, wherein said at least one additional compound is selected from the group consisting of caffeine, erythropoietin, magnesium sulfate, oxygen gas, dexamethasone, prednisone, and hydrocortisone.

10. The method of claim 1, wherein said patient is human.

11. The method of claim 10, wherein said patient is a premature infant.

12. A method of stimulating proliferation of a CNS cell in a patient in need thereof, wherein said method comprises administering to said patient a therapeutically effective amount of a pharmaceutical composition comprising a K_{ATP} channel activator, wherein said CNS cell is selected from the group consisting of pre-oligodendrocytes, oligodendrocyte stem cells and glia cells.

13. The method of claim 12, wherein said activator is selected from the group consisting of 7-chloro-3-methyl-4H-1,2,4-benzothiadiazine 1,1-dioxide, (E)-1-cyano-2-tert-pentyl-3-(pyridin-3-yl)guanidine, (3S,4R)-3-hydroxy-2,2-dimethyl-4-(2-oxopyrrolidin-1-yl)chroman-6-carbonitrile, (E)-1-(3,3-dimethylbutan-2-yl)-2-cyano-3-(pyridin-4-yl)guanidine, 3,3,3-trifluoro-2-hydroxy-2-methyl-N-(4-(phenylsulfonyl)phenyl)propanamide, N-((3S,4R)-6-cyano-3-hydroxy-2,2-dimethylchroman-4-yl)-N-hydroxyacetamide, and acceptable salts thereof.

14. The method of claim 13, wherein said activator is 7-chloro-3-methyl-4H-1,2,4-benzothiadiazine 1,1-dioxide or an acceptable salt thereof.

15. The method of claim 12, further comprising administering to said patient a therapeutically effective amount of at least one additional compound known to stimulate proliferation of said CNS cell.

16. The method of claim 15, wherein said at least one additional compound is selected from the group consisting of caffeine, erythropoietin, magnesium sulfate, oxygen gas, dexamethasone, prednisone, and hydrocortisone.

17. The method of claim 12, wherein said patient is human.

18. The method of claim 17, wherein said patient is a premature infant.


SEQUENCE LISTING (4 sequences)

SEQ ID NO: 1 - DNA, 4980 bp, Homo sapiens
cggggcccggggggcgggggcctgacggccgggccgggcggcggagctgcaagggacagaggcgcggcaggcgcgcggagccagcggagccagctgagcccgagcccagcccgcgcccgcgccgccatgcccctggccttctgcggcagcgagaaccactcggccgcctaccgggtggaccagggggtcctcaacaacggctgctttgtggacgcgctcaacgtggtgccgcacgtcttcctactcttcatcaccttccccatcctcttcattggatggggaagtcagagctccaaggtgcacatccaccacagcacatggcttcatttccctgggcacaacctgcggtggatcctgaccttcatgctgctcttcgtcctggtgtgtgagattgcagagggcatcctgtctgatggggtgaccgaatcccaccatctgcacctgtacatgccagccgggatggcgttcatggctgctgtcacctccgtggtctactatcacaacatcgagacttccaacttccccaagctgctaattgccctgctggtgtattggaccctggccttcatcaccaagaccatcaagtttgtcaagttcttggaccacgccatcggcttctcgcagctacgcttctgcctcacagggctgctggtgatcctctatgggatgctgctcctcgtggaggtcaatgtcatcagggtgaggagatacatcttcttcaagacaccgagggaggtgaagcctcccgaggacctgcaagacctgggggtacgcttcctgcagcccttcgtgaatctgctgtccaaaggcacctactggtggatgaacgccttcatcaagactgcccacaagaagcccatcgacttgcgagccatcgggaagctgcccatcgccatgagggccctcaccaactaccaacggctctgcgaggcctttgacgcccaggtgcggaaggacattcagggcactcaaggtgcccgggccatctggcaggcactcagccatgccttcgggaggcgcctggtcctcagcagcactttccgcatcttggccgacctgctgggcttcgccgggccactgtgcatctttgggatcgtggaccaccttgggaaggagaacgacgtcttccagcccaagacacaatttctcggggtttactttgtctcatcccaagagttccttgccaatgcctacgtcttagctgtgcttctgttccttgccctcctactgcaaaggacatttctgcaagcatcctactatgtggccattgaaactggaattaacttgagaggagcaatacagaccaagatttacaataaaattatgcacctgtccacctccaacctgtccatgggagaaatgactgctggacagatctgtaatctggttgccatcgacaccaatcagctcatgtggtttttcttcttgtgcccaaacctctgggctatgccagtacagatcattgtgggtgtgattctcctctactacatactcggagtcagtgccttaattggagcagctgtcatcattctactggctcctgtccagtacttcgtggccaccaagctgtctcaggcccagcggagcacactggagtattccaatgagcggctgaagcagaccaacgagatgctccgcggcatcaagctgctgaagctgtacgcctgggagaacatcttccgcacgcgggtggagacgacccgcaggaaggagatgaccagcctcagggcctttgccatctatacctccatctccattttcatgaacacggccatccccattgcagctgtcctcataactttcgtgggccacgtcagcttcttcaaagaggccgacttctcgccctccgtggcctttgcctccctctccctcttccatatcttggtcacaccgctgttcctgctgtccagtgtggtccgatctaccgtcaaagctctagtgagcgtgcaaaagctaagcgagttcctgtccagtgcagagatccgtgaggagcagtgtgccccccatgagcccacacctcagggcccagccagcaagtaccaggcggtgcccctcagggttgtgaaccgcaagcgtccagcccgggaggattgtcggggcctcaccggcccactgcagagcctggtccccagtgcagatggcgatgctgacaactgctgtgtccagatcatgggaggctacttcacgtggaccccagatggaatccccacactgtccaacatcaccattcgtatcccccgaggccagctgactatgatcgtggggcaggtgggctgcggcaagtcctcgctccttctagccgcactgggggagatgcagaaggtctcaggggctgtcttctggagcagccttcctgacagcgagataggagaggaccccagcccagagcgggagacagcgaccgacttggatatcaggaagagaggccccgtggcctatgcttcgcagaaaccatggctgctaaatgccactgtggaggagaacatcatctttgagagtcccttcaacaaacaacggtacaagatggtcattgaagcctgctctctgcagccagacatcgacatcctgccccatggagaccagacccagattggggaacggggcatcaacctgtctggtggtcaacgccagcgaatcagtgtggcccgagccctctaccagcacgccaacgttgtcttcttggatgaccccttctcagctctggatatccatctgagtgaccacttaatgcaggccggcatccttgagctgctccgggacgacaagaggacagtggtcttagtgacccacaagctacagtacctgccccatgcagactggatcattgccatgaaggatggcaccatccagagggagggtaccctcaaggacttccagaggtctgaatgccagctctttgagcactggaagaccctcatgaaccgacaggaccaagagctggagaaggagactgtcacagagagaaaagccacagagccaccccagggcctatctcgtgccatgtcctcgagggatggccttctgcaggatgaggaagaggaggaagaggaggcagctgagagcgaggaggatgacaacctgtcgtccatgctgcaccagcgtgctgagatcccatggcgagcctgcgccaagtacctgtcctccgccggcatcctgctcctgtcgttgctggtcttctcacagctgctcaagcacatggtcctggtggccatcgactactggctggccaagtggaccgacagcgccctgaccctgacccctgcagccaggaactgctccctcagccaggagtgcaccctcgaccagactgtctatgccatggtgttcacggtgctctgcagcctgggcattgtgctgtgcctcgtcacgtctgtcactgtggagtggacagggctgaaggtggccaagagactgcaccgcagcctgctaaaccggatcatcctagcccccatgaggttttttgagaccacgccccttgggagcatcctgaacagattttcatctgactgtaacaccatcgaccagcacatcccatccacgctggagtgcctgagccgctccaccctgctctgtgtctcagccctggccgtcatctcctatgtcacacctgtgttcctcgtggccctcttgcccctggccatcgtgtgctacttcatccagaagtacttccgggtggcgtccagggacctgcagcagctggatgacaccacccagcttccacttctctcacactttgccgaaaccgtagaaggactcaccaccatccgggccttcaggtatgaggcccggttccagcagaagcttctcgaatacacagactccaacaacattgcttccctcttcctcacagctgccaacagatggctggaagtccgaatggagtacatcggtgcatgtgtggtgctcatcgcagcggtgacctccatctccaactccctgcacagggagctctctgctggcctggtgggcctgggccttacctacgccctaatggtctccaactacctcaactggatggtgaggaacctggcagacatggagctccagctgggggctgtgaagcgcatccatgggctcctgaaaaccgaggcagagagctacgaggggctcctggcaccatcgctgatcccaaagaactggccagaccaagggaagatccagatccagaacctgagcgtgcgctacgacagctccctgaagccggtgctgaagcacgtcaatgccctcatcgcccctggacagaagatcgggatctgcggccgcaccggcagtgggaagtcctccttctctcttgccttcttccgcatggtggacacgttcgaagggcacatcatcattgatggcattgacatcgccaaactgccgctgcacaccctgcgctcacgcctctccatcatcctgcaggaccccgtcctcttcagcggcaccatccgatttaacctggaccctgagaggaagtgctcagatagcacactgtgggaggccctggaaatcgcccagctgaagctggtggtgaaggcactgccaggaggcctcgatgccatcatcacagaaggcggggagaatttcagccagggacagaggcagctgttctgcctggcccgggccttcgtgaggaagaccagcatcttcatcatggacgaggccacggcttccattgacatggccacggaaaacatcctccaaaaggtggtgatgacagccttcgcagaccgcactgtggtcaccatcgcgcatcgagtgcacaccatcctgagtgcagacctggtgatcgtcctgaagcggggtgccatccttgagttcgataagccagagaagctgctcagccggaaggacagcgtcttcgcctccttcgtccgtgcagacaagtgacctgccagagcccaagtgccatcccacattcggaccctgcccatacccctgcctgggttttctaactgtaaatcacttgtaaataaatagatttgattatttcctaaa

SEQ ID NO: 2 - DNA, 4670 bp, Homo sapiens
agtaaaccatataagaagaaatgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcctaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgttcctgctctccacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaacttgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttcgaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgttggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatagctcaccgtgtctcttctattatggatgcaggccttgttttagtcttttctgagggtattttagtggagtgtgatactgtcccaaatttgctcgcccacaagaatggcctcttttccactttggtgatgaccaacaagtag

SEQ ID NO: 3 - DNA, 2381 bp, Homo sapiens
ttccactgaagtggccaagggagttgagctgagcagggggaggggaagatctacacccatcccccttcctctagccctccctgcgtttagattcagttgcaccttttattattttaactcttctccttaggacacgcagcccccaatttgtccctccggcctgggcggcccctggtcccgcgcgccagcatgggagagcgagggacctgcccgcggcccgccggcgtgtgcaaggaggtccagccgccgcgcccgctacccggagtctgaggacgggtgtccagggacggagaggcaggtgagagggaggtggctaagctggctatggtgacaggacgatgttggccagaaagagtatcatcccggaggagtatgtgctggcgcgcatcgccgcagagaacctgcgcaagccgcgcatccgagaccgcctccccaaagcccgcttcatcgccaagagcggggcctgcaacctggcgcataagaacatccgtgagcaaggacgctttctacaggacatcttcaccaccttggtggacctgaaatggcgccacacgctggtcatctttaccatgtccttcctctgcagctggctgctcttcgctatcatgtggtggctggtggcctttgcccatggggacatctatgcttacatggagaaaagtggaatggagaaaagtggtttggagtccactgtgtgtgtgactaatgtcaggtctttcacttctgcttttctcttctccattgaagttcaagttaccattgggtttggagggaggatgatgacagaggaatgccctttggccatcacggttttgattctccagaatattgtgggtttgatcatcaatgcagtcatgttaggctgcattttcatgaaaacagctcaggctcacagaagggcagaaactttgattttcagccgccatgctgtgattgccgtccgaaatggcaagctgtgcttcatgttccgagtgggtgacctgaggaaaagcatgatcattagtgcctctgtgcgcatccaggtggtcaagaaaacaactacacctgaaggggaggtggttcctattcaccaactggacattcctgttgataacccaatcgagagcaataacatttttctggtggcccctttgatcatctgccacgtgattgacaagcgcagtcccctgtatgacatctcagcaactgacctggccaaccaagacttggaggtcatagttattctggaaggagtggttgaaactactggcatcaccacacaagcacgaacctcctacattgctgaggagatccaatggggccaccgctttgtgtccattgtgactgaggaagaaggagtgtattctgtggattactccaaatttggcaacactgttaaagtagctgctccacggtgcagtgcccgagagctggatgagaaaccttccatccttattcagaccctccaaaagagtgaactgtctcatcaaaattctctgaggaagcgcaactccatgagaagaaacaattccatgaggaggaacaattctatccgaaggaacaattcttccctcatggtaccaaaggtgcaatttatgactccagaaggaaatcaaaacacatcggaatcatgacagcaagataaccccaagacagtcttttatcaagttttgacggtttatgctgggcactgccagactgaaccagagctggaacacaataatgtgtccttctacattttattacactaaatgatattcatattcaagcaacagcactttctgtagtaataaaaagtaacacaccaagtggaggattcatggcttatgcttgtttcatacatgtagaattcgcagtgatatacttaagttatgtgcaggggtgggtatgctcgtttctctcaatttgaaacacaagaattacactaatagtaacaggccttgagcacaaaatttggtcaagaaatgagtctgtttgaaatgcttagttggtgaaacccagatcgctgtgttactaatgatcactctggaaatgcaaatagaataggaggggaaaaaacaaaatcatgttaatgcgttttgtacatcagccttctcctgtctccatgataactgcatttggtaggaagcctgaggcaaaaactgcttaagtggatctgtttgtaagtggattcagttcattcgctttgtctgtcggtgactcctgaaggtttcctggtccatctcattgcggcctgttgggcagagctggacaaggtgtctttgaaggaaagctaagtcttgtctcatttaggactgggttccccacagtgacagtcacttccttcagcatctcacttcttcttctagaatccccacagggaaatactgtattgaatattttattaaaacattttctttgta

SEQ ID NO: 4 - DNA, 3418 bp, Homo sapiens
ggcagggttgggggctccctaggcgccaggcaggtgggctcaagggtgaggctgttttttttgttttgttttttgtttttgagacggagtctcgctctgtcgcccaggctggagtgcagtggcgtgatcttggctcactgcaacctccgcctctcgggttcaagcgattctcctgcctcagcttcctgagtagctgggattacaggtgcgcaccaccatgcccggctaacttttgtatttttagtagagatggggtttcaccatgttggtcaggctggtctcgaactcctgacctagtgatctgccctcctcagcctcccaacgtactgggattacaggcgtgagccaccgcgcccggcctgaggctggtattaagaagtgaagtgggacccaggtggaggtaaggaagagtctggtggggagttatctcagaagtgaggccagcacaggctgagtgcagccccagggtgagaaggtgcccaccgagaggactctgcagtgaggccctaggccacgtccgaggggtgcctccgatgggggaagcccctccctgggggtcaccggagccatgctgtcccgcaagggcatcatccccgaggaatacgtgctgacacgcctggcagaggaccctgccaagcccaggtaccgtgcccgccagcggagggcccgctttgtgtccaagaaaggcaactgcaacgtggcccacaagaacatccgggagcagggccgcttcctgcaggacgtgttcaccacgctggtggacctcaagtggccacacacattgctcatcttcaccatgtccttcctgtgcagctggctgctcttcgccatggcctggtggctcatcgccttcgcccacggtgacctggcccccagcgagggcactgctgagccctgtgtcaccagcatccactccttctcgtctgccttccttttctccattgaggtccaagtgactattggctttggggggcgcatggtgactgaggagtgcccactggccatcctgatcctcatcgtgcagaacatcgtggggctcatgatcaacgccatcatgcttggctgcatcttcatgaagactgcccaagcccaccgcagggctgagaccctcatcttcagcaagcatgcggtgatcgccctgcgccacggccgcctctgcttcatgctacgtgtgggtgacctccgcaagagcatgatcatcagcgccaccatccacatgcaggtggtacgcaagaccaccagccccgagggcgaggtggtgcccctccaccaggtggacatccccatggagaacggcgtgggtggcaacagcatcttcctggtggccccgctgatcatctaccatgtcattgatgccaacagcccactctacgacctggcacccagcgacctgcaccaccaccaggacctcgagatcatcgtcatcctggaaggcgtggtggaaaccacgggcatcaccacccaggcccgcacctcctacctggccgatgagatcctgtggggccagcgctttgtgcccattgtagctgaggaggacggacgttactctgtggactactccaagtttggcaacaccgtcaaagtgcccacaccactctgcacggcccgccagcttgatgaggaccacagcctactggaagctctgaccctcgcctcagcccgcgggcccctgcgcaagcgcagcgtgcccatggccaaggccaagcccaagttcagcatctctccagattccctgtcctgagccatggtctctcgggccccccacacgcgtgtgtacacacggaccatgtggtatgtagcccggccagggcctggtgtgaggctgggccagcctcagctcagcctccccctgctgctcatccagggtgttacaaggcacttgtcactatgctatttctggcctcagcaggaacctgtactgggttatttttgtccctgctcctcccaacccaattcaggactggctcacccctctcccccgcccaaggctgcagaggctgtgggaggtactgggccctagagctgtgcgtccagccagtcctgggtccccacgattgaccagccacactctgggccggtggctggggaagaacaatccccgagggctgctgctttgcgtctgtggctccaagaagtgcctgtggtcaggccccagctctacttggtccctgaaaaagcacctggctaagggctgggcctggccagcagggagggcagttgatgagagagggtgttcccgctggagggttggtgctgtggagcctacaccggcagggacagcctggggctgacagggctcccctccgagggccagtttcaggtctggaaggggaggaagcaggggaaggtgacctgaggaggctcggctttgtagagccccgctcaggcacagggaggaggagatgccagggctcctgccttttgccacatcggcctcgtgcagtgagggctctgtgggctggggctgctgcccctgcctacctcctgcctgtccccagaggctgaggagagggggtactgtgcccaccacacatgattaggcctcagacccaactctggtcctggctccacaacagtggctgccactcactttgtccagaaggtggcttgggggtggatatctttgggttgctggaaaaggtgtgggaaggttcaggatggtgggagggactgaggtccctgaggtgaagaggcccttggtcctgacgggtttgacccgtgcctggacccttggagcagtgttgtgtgaacttgcctagaactctgccttctccgttgtcaataaagcctccccctcatgacctaaactctgggcttttcttgctggggaggcagcaagcatgctggtgggaagggaggcagggactggcagctgccacccccttcaagaggcgccatagaccctagcggggagggcaggggagggacggaaggctggcacctcttccaccagttcagggggactttcccctctcctgtctcaggtggcccagccctgtcagcctgtctggccaactcagcctttgggcactcaccaggctttgcagccctgggctctgtctctactcccagggacctgctggaaggctggagtgcccagggagaggtatagaggtgtcataggcattagtgtagtaattggagcactaactctcgagccaactgcctgggttcgaatcctggctctagctgtatgacttttgtcaagtaacttagcctctctgtgtctcagttgcctcttctataacatggatgctaatagtacctacctcatagaattgttttggaagtaaatgaaaaatatgtaaaatgctgaagtgcctggtctacagtaagtgctcaataaatgttaactattgtgattgctgctgaatcagctacatgctgaggaaacggccaaacaagtgttaaaaaaaaa